(19) United States
(12) Patent Application Publication
(10) Pub. No.: US 2021/0290733 A1
(43) Pub. Date: Sep. 23, 2021

(54) INJECTABLE MICRONIZED HUMAN INSULIN

(71) Applicant: ARVEN ILAC SANAYI VE TICARET ANONIM SIRKETI, Istanbul (TR)

(72) Inventors: Devrim CELIK, Istanbul (TR); Irem YENICE, Istanbul (TR); Ali Ihsan SECKIN, Istanbul (TR); Turgay KACAR, Istanbul (TR)

(73) Assignee: ARVEN ILAC SANAYI VE TICARET ANONIM SIRKETI, Istanbul (TR)

(21) Appl. No.: 17/259,904

(22) PCT Filed: Jul. 11, 2019
(86) PCT No.: PCT/TR2019/050561

§ 371 Date: Jan. 12, 2021

(30) Foreign Application Priority Data
Jul. 16, 2018 (TR) 2018/10086

(51) Int. Cl. A61K 38/28; A61K 9/00; A61K 9/10; A61K 9/14

(52) U.S. Cl. CPC A61K 38/28; A61K 9/141; A61K 9/10; A61K 9/0019

(57) ABSTRACT

The present invention relates to the micronized human insulin using injectable, for the treatment of types I and II diabetes C in mammalians, particularly in humans

### FIELD OF THE INVENTION

The present invention relates to the micronized human insulin using injectable, for the treatment of types I and II diabetes in mammalians, particularly in humans.

### BACKGROUND OF THE INVENTION

Diabetes mellitus is a group of disorders of carbohydrate metabolism in which the action of insulin is diminished or absent through altered secretion, decreased insulin activity or a combination of both factors. There are two main types of diabetes; Type 1 and Type 2:

Type 1 diabetes occurs because the insulin-producing cells of the pancreas (beta cells) are damaged. In Type 1 diabetes, the pancreas produces little or no insulin, so sugar cannot get into the body's cells for use as energy.

In Type 2 diabetes, the pancreas produces insulin, but it either doesn't produce enough, or the insulin does not work properly.

The goal of diabetes treatment is to keep blood glucose levels as close to normal as safely possible. There are several different ways for treatment of diabetes. The most common of these are insulin (human insulin and insulin analogs) or adjuvant (non-insulin) injectable or oral antidiabetic formulation. The purpose of these treatments is that improve the effectiveness of the body's natural insulin, reduce blood sugar production, increase insulin production and inhibit blood sugar absorption.

However, the use of these adjuvant (non-insulin) injectable or oral antidiabetic formulation is associated with various adverse effects, especially have side effects that are dependent on the excipient used.

Human insulin and insulin analogues are preferred and used by most adults with type 1 diabetes.

Insulin analogs are analogs that have been designed to mimic the body's natural pattern of insulin release. These synthetic-made insulins are called analogs of human insulin. However, they have minor structural or amino acid changes. Once absorbed, they act on cells like human insulin, but are absorbed from fatty tissue more predictably and the duration of action of these insulin analogs may be shorter. The activity of insulin analogs is not well synchronized with your body's needs.

Human insulin consists of two polypeptide chains, the A and B chains which contain 21 and 30 amino acid residues. The A and B chains are interconnected by two disulfide bridges. Insulin from most other species is similar but may contain amino acid substitutions in some positions.

Human insulin is marketed under the brand name Huminsulin® by Eli Lilly is a biphasic isophane insulin injection. It comprises soluble insulin, isophane insulin and pharmaceutical acceptable excipients which are preservative, tonicity modifier, pH adjustment agent. Also, other medicinal product Mixtard® marketed by Novo Nordisk is a dual-acting, biphasic formulation comprises a premix of soluble fast-acting insulin human, isophane long-acting insulin and at least one pharmaceutical acceptable excipients.

Human insulin is identical in structure to your own natural insulin. But when it is injected under the skin it doesn't work as well as natural insulin. This is because injected human insulin clumps together and takes a long time to get absorbed.

Therefore, there is still need for new approaches in order to develop injectable composition comprising human insulin which overcomes the above described problems in the prior art and has additive advantages over them.

In this present study, the effect of using injectable micronized human insulin was studied in the treatment of diabetes mellitus. In comparison to the prior art, it was observed that micronized human insulin was absorbed easily and fast in a body, providing effective treatment and high bioactivity for injectable use.

### DETAILED DESCRIPTION OF THE INVENTION

The main object of the present invention is to provide micronized human insulin for treating diabetes mellitus which improve the pharmacokinetics and pharmacodynamics of injectable human insulin compositions by increasing the rate of absorption from the site of subcutaneous injection.

The object of the present invention is to have high bioactivity and effective treatment without any side effect.

The object of the present invention is to provide micronized human insulin having a long-term stability.

The object of the present invention is to provide a method to obtain micronized human insulin.

Human insulin in the present invention is an unmodified insulin. In terms of the invention, the human insulin is a recombinant human insulin which has not undergone any transformation of its primary structure or any modification of the side groups of the amino acids.

The term “micronization”, as used herein, refers to a process used in the pharmaceutical industry that reduces the particle size of human insulin.

According to one embodiment of the present invention, the injectable composition comprises micronized human insulin.

According to one embodiment of the present invention, the composition is prepared by based on USP 121 formula (The United States Pharmacopeial Convention 2015, [121] Insulin Assays). This is important in order to minimize the risk of adverse effects, both local and systemic. It is important that human insulin in the composition is used without extra excipients because, its amino acid sequence and biological activity are the same as those of the human body and are safe and have no toxic or side effects.

According to one embodiment of the present invention, after micronization, the micronized human insulin has a d(0.1) particle size which is less than 5 μm or less than 3 μm or less than 2 μm.

According to one embodiment of the present invention, after micronization, the micronized human insulin has a d(0.5) particle size which is less than 9 μm or less than 7 μm or less than 6 μm.

According to one embodiment of the present invention, after micronization, the micronized human insulin has a d(0.9) particle size which is less than 16 μm or less than 14 μm or less than 13 μm.

After micronization, the micronized human insulin has a d(0.1) particle size which is less than 5 μm, a d(0.5) particle size which is less than 9 μm, a d(0.9) particle size which is less than 16 μm.

After micronization, the micronized human insulin has a d(0.1) particle size which is less than 3 μm, a d(0.5) particle size which is less than 7 μm, a d(0.9) particle size which is less than 14 μm.

After micronization, the micronized human insulin has a d(0.1) particle size which is less than 2 μm, a d(0.5) particle size which is less than 6 μm, a d(0.9) particle size which is less than 13 μm.

It has surprisingly been found that improved pharmacokinetic and pharmacodynamic properties and easy and fast absorption in the body can be obtained when human insulin becomes micronized. The particle sizes mentioned above increases the paracellular permeability of human insulin and provides effective treatment and high bioactivity for injectable use. Also, it provides long-time stability.

According to one embodiment of the present invention, the micronized human insulin particles were prepared with a technique of size reduction.

Some important techniques of micronization are temperature control technique, subsequent treatment technique, wet and dry milling, sono-crystallization, spray drying or supercritical fluid process.

The working principle of the temperature control technique, comprises to use only a small percentage of the energy of milling to form new surface, the bulk of the energy is converted to heat so reduced size is obtained. The subsequent treatment technique is used if extreme control of size is required, the working principle of it comprises to recycle the larger particles, either by simply screening the discharge and returning the oversize particles for a second milling or by using air separation equipment.

The choice of dry or wet milling depends on the use of the product and its subsequent processing. If the product undergoes physical or chemical change in water, dry milling is recommended. In dry milling, the limit of fineness is reached in the region of 100 microns when the material cakes on the milling chamber. The addition of a small amount of grinding aid may facilitate size reduction.

The working principle of sono-crystallisation comprises utilizing ultrasound power characterized by a frequency range of 20-100 kHz for inducing crystallization. It not only enhances the nucleation rate but also an effective means of size reduction and controlling the size distribution of the active pharmaceutical ingredients is provided.

Spray drying is a commonly used method of drying a liquid feed through a hot gas. Typically, this hot gas is air but sensitive materials such as pharmaceuticals and solvent like ethanol require oxygen-free drying and nitrogen gas is used.

The supercritical fluid technology is a high-efficiency method of preparing micronized human insulin having a particle size at the micrometer level suitable for a pharmaceutical injectable.

Supercritical fluid technology (SOFT) has been used in many fields for decades, such as the food industry, chemical processing, polymers, textile, forest product industries, and in the pharmaceutical field. In the pharmaceutical field, the technology may be used for the reduction of particle size and designing of novel drug delivery systems.

Supercritical fluids (SCFs) possess properties that are intermediate between liquids and gases. This unique phase is obtained through the exertion of pressures and temperatures greater than the critical point (FIG. 1). Near the critical point of a fluid, minor changes in pressure or temperature significantly alter the physicochemical properties of the SCF (e.g., density, diffusivity, or solubility characteristics).

It can be defined as a dense non-condensable fluid. Supercritical fluids are fluids whose temperature and pressure are greater than its critical temperature (Tc) and critical pressure (Tp). SCFs are high compressible, allowing moderate changes in pressure to greatly alter the density and mass transport characteristics of fluid that largely determine its solvents power.

Drug particles are solubilized within SCFs, they may be recrystallized at greatly reduced particle sizes.

The most commonly used supercritical fluid for a variety of applications is carbon dioxide, ethane, propane, ammonia, methanol, n-pentane, toluene or water.

According to this embodiment of the present invention, carbon dioxide is used as the supercritical fluid. Carbon dioxide as a solvent has many advantages.
It is non-toxic, not flammable and inexpensive.
Critical temperature and pressure of it is low. (critical pressure Pc=73.8 bar, critical temperature Tc=31.1° C.)
Carbon dioxide exhibits high solvency for non-polar and medium-polar organic compounds.
Carbon dioxide is stable against chemicals and radioactive substances.
Carbon dioxide is easily separated from the system.

Classification of SCFT based techniques can be proposed according to the role played by the SCFT in the process. Various SCFT processes used in pharmaceutical processing include;
1. Rapid expansion of supercritical solutions (RESS),
2. Supercritical antisolvent (SAS) precipitation technique.

Typically, in a SAS process, the material is dissolved in a solvent and then sprayed into a high-pressure chamber filed with supercritical carbon dioxide. As each droplet is formed in the high-pressure vessel, the carbon dioxide extracts the solvent, leaving behind a powder with low bulk density. This technique is used in the present invention.

Suitable solvents in this technique are selected from the group comprising dimethyl sulfoxide, ethanol, hexafluoro-2-propanol, acetone, dichloromethane, methanol, chloroform, ethyl acetate, dimethylformamide, diethyl sulfoxide, diethyl ether, tetrahydrofuran, hexanes. Preferably solvent in this technique is dimethyl sulfoxide, ethanol or hexafluoro-2-propanol or mixtures thereof.

The reduced particle size was attained through the choice of the proper supercritical fluid process and the optimization of processing parameters.

According to one embodiment of the present invention, the micronized human insulin particles were prepared by using supercritical fluid process.

According to one embodiment of the present invention, the pharmaceutical composition is administered through subcutaneous, intravenous or intramuscular route to a subject, and wherein the pharmaceutical composition is for use in the treatment of type I and type II diabetes mellitus. Preferably, the pharmaceutical composition is administered through subcutaneous route.

According to one embodiment of the present invention, the human insulin is prepared by using the supercritical fluid process is characterized in that the prepared human insulin is for using injectable with a d (0.1) particle size of less than 5 μm, a d (0.5) particle size of less than 9 μm, a d (0.9) particle size of less than 16 μm for use in the treatment of type I and type II diabetes mellitus.

TABLE 1
The particle size of the human insulin measurement result

| | Before Micronization | | | After Micronization | | |
|---|---|---|---|---|---|---|
| | d(0.1) | d(0.5) | d(0.9) | d(0.1) | d(0.5) | d(0.9) |
| Particle | 1.96 | 13.24 | 25.12 | 1.654 | 5.273 | 12.54 |
| size (μm) | | | | | | |

The particle size is a critical parameter that determines the rate of absorption of human insulin and thus has significant effect on bioavailability.

TABLE 2
The result of bioactivity test of micronized human insulin
Bioactivity Test

| USP Limit | Before Micronization | After Micronization |
|---|---|---|
| min. 15 IU/mg | 16.02 IU/mg | 34.72 IU/mg |

According to the bioactivity test, when the results were examined, it was seen that protein activity increased at the end of the micronization process without any change in protein structure. This increase shows that the micronized human insulin passes faster into the blood than un-micronized human insulin.

1. An injectable composition comprising micronized human insulin.

2. The injectable composition according to claim 1, wherein the micronized human insulin has a d(0.1) particle size which is less than 5 μm.

3. The injectable composition according to claim 1, wherein the micronized human insulin has a d(0.5) particle size which is less than 9 μm.

4. The injectable composition according to claim 1, wherein the micronized human insulin has a d(0.9) particle size which is less than 16 μm.

5. The injectable composition according to claim 1, wherein the micronized human insulin has a d(0.1) particle size which is less than 5 μm, a d(0.5) particle size which is less than 9 μm, a d(0.9) particle size which is less than 16 μm.

6. The injectable composition according to claim 1, wherein the micronized human insulin has a d(0.1) particle size which is less than 3 μm, a d(0.5) particle size which is less than 7 μm, a d(0.9) particle size which is less than 14 μm.

7. The injectable composition according to claim 1, wherein the techniques of micronization of human insulin comprising temperature control technique, subsequent treatment technique, wet and dry milling, sono-crystallization, spray drying or supercritical fluid process.

8. The injectable composition according to claim 7, wherein micronization is provided by using the supercritical fluid process.

9. The injectable composition according to claim 1, wherein the composition is administered through subcutaneous, intravenous or intramuscular route.

10. The injectable composition according to claim 9, wherein the composition is administered through subcutaneous route.

11. The injectable composition according to claim 1, for use in the treatment of diabetes mellitus.
